(19) United States
(12) Patent Application Publication
(10) Pub. No.: US 2024/0395371 A1
(43) Pub. Date: Nov. 28, 2024

(54) METHOD AND SYSTEM FOR MANAGEMENT OF MEDICAL CARE RESOURCES

(71) Applicant: A3P BIOMEDICAL AB (PUBL), Stockholm (SE)

(72) Inventors: Karl ANDERSSON, Vange (SE); Martin EKLUND, Uppsala (SE)

(21) Appl. No.: 18/694,603

(22) PCT Filed: Sep. 29, 2022
(86) PCT No.: PCT/EP2022/077100

§ 371 Date: Mar. 22, 2024

(30) Foreign Application Priority Data
Oct. 1, 2021 (SE) 2151208-2

(51) Int. Cl. G16H 10/40; G16H 40/20

(52) U.S. Cl. CPC G16H 10/40; G16H 40/20

(57) ABSTRACT

A method (100) and a system for management of medical care resources are provided. The method comprises the steps of collecting (110) statistical information (120) associated with screening tests (130) for a disease, wherein the statistical information is associated with at least one classification (150) of an individual of a group of individuals, defining (160) a group of persons having an estimated need for one of the screening tests according to the classification, establishing (170) a first level of availability of the at least one treatment resource, selecting (180) a screening test to be used for the defined group of persons based on the statistical information under a boundary condition that the average of the estimated need of the at least one treatment resource is not allowed to exceed the first level of availability of the at least one treatment resource.

### TECHNICAL FIELD

The present invention relates in general to methods and systems for management of medical care resources. In particular, the present invention relates to the management of such resources associated with the selection of a screening test for a disease.

### BACKGROUND

The management of medical care resources in institutions such as hospitals, clinics, health centers, etc., is of vital importance for optimizing the care of patients within the constraints of the resources available (e.g. medical staff, patient rooms, etc.).

The management of the resources for the medical care of patients often suffers from an inferior planning. In case of a relatively high strain on the health care institution(s) due to factors such as a high number of patients, limited staffing capabilities, etc., the effects of such deficient management and/or planning for the patients can be severe. There is also a need to manage the working conditions for the staff such as nurses, doctors, etc., in order to ensure a sustainable and functional working environment.

The medical care resource management is strongly linked to screening tests of the patients for a disease. Notably, in case of two or more available test, there is a wish to select the most suitable screening test under the restrictions of the medical care resources associated with the disease that the screening test indicates.

The most flagrant example of the importance of medical care resource management may be taken from the recent COVID-19 pandemic, leading to a vast number of patients during a relatively short period of time.

Another example of the importance of medical care resource management is related to screening and treatment of cancer, such as prostate cancer, being the second most common cancer in the world with approximately 1.4 million men being diagnosed yearly.

Hence, there is a wish to achieve an improved management of medical care resources by improving the selection of screening test of the patient.

### SUMMARY

The above object is achieved by methods and systems according to the independent claims. Preferred embodiments are defined in dependent claims.

According to a first aspect of the present invention, there is provided a method for management of medical care resources. The method comprises the step of collecting statistical information associated with at least two screening tests for a disease, wherein the statistical information comprises average of an estimated need of at least one treatment resource for treatment of the disease, after a final conclusive diagnosis of the disease has been obtained, wherein the statistical information is associated with at least one classification of an individual of a group of individuals. The method further comprises the step of defining a group of persons having an estimated need for one of the at least two screening tests according to the classification. The method further comprises the step of establishing a first level of availability of the at least one treatment resource. The method further comprises the step of selecting a screening test of the at least two screening tests to be used for the defined group of persons based on the statistical information under a boundary condition that the average of the estimated need of the at least one treatment resource is not allowed to exceed the first level of availability of the at least one treatment resource.

According to a second aspect of the present invention, there is provided a system for management of medical care resources. The system comprises an input configured to receive statistical information associated with at least two screening tests for a disease, the statistical information comprising average of an estimated need of at least one treatment resource for treatment of the disease, after a final conclusive diagnosis of the disease has been obtained, wherein the statistical information is associated with at least one classification of an individual of a group of individuals, information associated with a group of persons having an estimated need for one of the at least two screening tests according to the classification, and a first level of availability of the at least one treatment resource. The system is configured to select a screening test of the at least two screening tests based on the statistical information under a boundary condition that the average of the estimated need of the at least one treatment resource is not allowed to exceed the first level of availability of the at least one treatment resource.

Thus, the present invention is based on the idea of providing a method for management of medical care resources based on the selection of a screening test, among two or more screening tests, to be used for a defined group of persons. The screening test selection, which is performed under a boundary condition of the estimated need of treatment resources for the disease, is based on statistical information of the estimated need for treatment resources for individuals having need of one of the screening tests.

The present invention is advantageous in that a screening test may be efficiently and conveniently selected based on statistical information of treatment resource(s) for treatment of the disease, wherein the statistical information is associated with classification(s) of an individual. In other words, the method is efficient and convenient in its screening test selection for a defined group of persons as it uses knowledge and/or estimates of the treatment resource(s) needed for treatment of the disease associated with classification(s) of individual(s).

The present invention is advantageous in that, among two or more screening tests to choose from, the most suitable screening test may be selected for the defined group of persons under the defined boundary conditions, which in turn leads to an optimization of the medical care resources.

The present invention is further advantageous in that the screening test selection may improve the planning of the treatment of the defined group of persons. This, in turn, may improve the rate of success of the treatment of the defined group of persons. Furthermore, the improved planning may improve working conditions for medical staff such as nurses, doctors, etc., in order to ensure a sustainable and functional working environment.

The present invention is further advantageous in that a screening test may be used that minimizes the spending of medical resources (like pathology and treatment resources) while finding relevant cancers. The screening test selection may not only lead to direct savings of medical resources, but may consequently also lead to an improved management of care resources in a manner that may extend the lifetime of persons, leading to savings for society both in terms of human suffering and in terms of economy.

There is provided a method for management of medical care resources. By the term “medical care resources”, it is meant e.g. personnel and/or equipment for medical care and/or treatment of patient(s). The method comprises the step of collecting statistical information associated with at least two screening tests for a disease. Hence, the method may be configured to collect and/or receive statistical information which is associated or linked to two or more screening tests for a disease. By the term “screening test”, it is here meant a test for identifying one or more conditions of an individual, which condition(s), possibly, could develop into a disease. The statistical information comprises an average of an estimated need of at least one treatment resource for treatment of the disease after a final conclusive diagnosis of the disease has been obtained. In other words, the statistical information comprises an average of an estimated or predicted need or requirement of one or more treatment resources when a conclusive diagnosis or confirmation of the disease has been obtained. The statistical information is associated with at least one classification of an individual of a group of individuals. By the term “classification”, it is here meant a category, characteristic, property and/or attribute. Consequently, by the wording “classification of an individual of a group of individuals”, it is here meant a category, characteristic, property and/or attribute of an individual of a group of individuals. The method further comprises the step of defining a group of persons having an estimated need for one of the at least two screening tests according to the classification. In other words, the step of the method comprises defining or selecting a group of persons having an estimated or predicted need for one screening test of the two or more screening tests. The method further comprises the step of establishing a first level of availability of the at least one treatment resource. Hence, the step of the method concerns establishing, defining or determining a first level of the availability or disposal of the treatment resource(s). The method further comprises the step of selecting a screening test of the at least two screening tests to be used for the defined group of persons based on the statistical information under a boundary condition that the average of the estimated need of the at least one treatment resource is not allowed to exceed the first level of availability of the at least one treatment resource. In other words, the method comprises the step of selecting a screening test for the defined group of persons based on, or as a function of, the statistical information, under the boundary condition that the average of the estimated or predicted need is maintained below, or amounts to, the first level of availability of the treatment resource(s).

According to an embodiment of the present invention, the average of an estimated need of at least one treatment resource for treatment of the disease may include time expenditure of the at least one treatment resource.

According to an embodiment of the present invention, the at least one classification may comprise at least one of a state of health of the individual and at least one factor selected from the group consisting of the gender of the individual, the age of the individual, the years of education of the individual, the birth country of the individual, the ethnicity of the individual, dietary habits of the individual, the income of the individual and the civil status of the individual. Hence, the classification(s) of an individual of the group of individuals may comprise a state of health of the individual, wherein the state of health may be based on one or more factors, attributes, personal information and/or indicators related to the individual's body such as body mass index, BMI, blood pressure, etc., in combination with, or solely based on one or more factors and/or indicators related to the individual's background, state of living, etc., such as gender, age, education, birth country, ethnicity, dietary habits, income and/or civil status. The present embodiment is advantageous in that the classification of the individual may become more detailed and/or customized, which in turn may provide an even more precise estimation of the need of the treatment resource(s). In turn, this may even further improve the selection of screening test, as the statistical information is associated with the classification.

According to an embodiment of the present invention, the at least one treatment resource may comprise at least one of medical staff for treatment of the defined group of persons, medical equipment for treatment of the defined group of persons, and pharmaceuticals for treatment of the defined group of persons. Hence, the treatment resource(s) for treatment of the disease may include medical staff, medical equipment and/or pharmaceuticals. The present embodiment is advantageous in that the selection of the screening test may be even further improved by the knowledge of the available resource(s). For example, if one or more of the mention resources is scarce, i.e. that it is established that the (first) level of availability of the treatment resource(s) is scarce, the most suitable screening test may be selected based on the low (restricted) level of availability of the treatment resource(s).

According to an embodiment of the present invention, the step of selecting a screening test of the at least two screening tests may be performed by minimizing a cost function, the cost function comprising terms being dependent on at least one of: economical costs for at least one additional test of the defined group of persons; economical costs for the at least one treatment resource; number of expected deaths from the disease within the defined group of persons having potential need for the screening test; and data associated with at least one of physical suffering of persons within the defined group of persons, and psychological suffering of persons within the defined group of persons, during at least one of at least one additional test of the disease of persons within the defined group of persons, and treatment of the disease of persons within the defined group of persons. Hence, the step of selecting the screening test may be performed by minimizing a cost function, wherein the cost function is dependent on economical costs for one or more additional tests of the defined group of persons and/or for the treatment resource(s), the number of expected deaths from the disease within the defined group of persons having potential need for the screening test, and/or data associated with physical and/or psychological suffering during one or more additional tests and/or treatment of the disease. The present embodiment is advantageous in that the selection of the screening test may be even further improved, as the screening test selection is based on the statistical information as well as the minimization of the factors related to cost, expected death and/or suffering related to additional test(s) and/or treatment of the disease.

According to an embodiment of the present invention, the disease may be caused by a virus.

According to an embodiment of the present invention, the disease may be COVID-19. It will be appreciated that the present invention is particularly advantageous for a disease such as COVID-19, as the recent pandemic, leading to a vast number of patients during a relatively short period of time, has emphasized the importance of medical care resource management. Hence, for a virus-caused disease such as COVID-19, the embodiment of the present invention may conveniently and efficiently select a screening test based on the statistical information comprising the need of treatment resource(s) for COVID-19 treatment.

According to an embodiment of the present invention, one of the screening tests may be a polymerase chain reaction, PCR, test. The present embodiment is advantageous in that PCR tests are very accurate when properly performed by a health care professional.

According to an embodiment of the present invention, one of the screening tests may be a lateral flow test.

According to an embodiment of the present invention, the statistical information may further comprise average of an estimated need of at least one additional test to the at least two screening tests, before a final conclusive diagnosis of the disease has been obtained, and wherein the method further comprises the steps of establishing a second level of available at least one additional test to the at least two screening tests, and selecting a screening test of the at least two screening tests to be used for the defined group of persons based on the statistical information under a boundary condition that the average of the estimated need of at least one additional test to the at least two screening tests and the average of the estimated need of the at least one treatment resource is not allowed to exceed the first level of availability of the at least one treatment resource, and the second level of available additional tests to the at least two screening test, respectively. In other words, the statistical information may additionally comprise an estimated need of additional/complementary test(s) (e.g. biopsy(ies)) to the screening test(s), and the selection of the screening test is based on the statistical information under a boundary condition that the average of the estimated need of additional test(s) to the screening tests and the average of the estimated need of the treatment resource(s) is not allowed to exceed the first level of availability of the treatment resource(s), and not allowed to exceed the second level of available additional tests to the screening tests, respectively. The present embodiment is advantageous in that the screening test selection may be refined and/or customized to an even higher extent, by taking into account the first level of availability of the treatment resource(s) as well as the second level of available additional tests.

According to an embodiment of the present invention, the disease may be cancer.

According to an embodiment of the present invention, the disease may be prostate cancer. It will be appreciated that the present invention is particularly advantageous for a disease such as prostate cancer, as prostate cancer is the second most common cancer in the world with approximately 1.4 million men being diagnosed yearly.

According to an embodiment of the present invention, one of the screening tests may be a blood test comprising an evaluation of a function of a combination of protein markers, genetic markers and clinical data. According to another embodiment, one of the screening tests may be a blood test which measures the amount of prostate specific antigen, PSA. Hence, one of the screening test for prostate cancer may be a blood test comprising an evaluation of a function of a combination of protein markers, genetic markers and clinical data, whereas one of the screening test for prostate cancer may be a PSA test. The blood test comprising an evaluation of a function of a combination of protein markers, genetic markers and clinical data, also denoted Stockholm3® (or STHLM3), is particularly advantageous in that is may detect (aggressive) prostate caner at an early stage. It will be appreciated that early detection is the key to successful treatment of prostate cancer. The PSA blood test is commonly used, but one problem with the PSA test is that it may miss between 30 and 50 percent of all aggressive cases of prostate cancer. In contrast, research has shown that Stockholm3® may find 100 percent more aggressive cases of cancer. Hence, the Stockholm3® blood test may improve the ability to detect and treat prostate cancer at an early stage.

According to an embodiment of the system of the present invention, the average of an estimated need of at least one treatment resource for treatment of the disease may include time expenditure of the at least one treatment resource.

According to an embodiment of the system of the present invention, at least one classification may comprise at least one of a state of health of the individual, and at least one factor selected from the group consisting of the gender of the individual, the age of the individual, the years of education of the individual, the birth country of the individual, the ethnicity of the individual, the dietary habits of the individual, the income of the individual and the civil status of the individual.

According to an embodiment of the system of the present invention, at least one treatment resource comprises at least one of medical staff for treatment of the defined group of persons, medical equipment for treatment of the defined group of persons, and pharmaceuticals for treatment of the defined group of persons.

According to an embodiment of the present invention, the system may further be configured to select a screening test of the at least two screening tests by minimizing a cost function, the cost function comprising terms being dependent on at least one of: economical costs for at least one additional test of the defined group of persons; economical costs for the at least one treatment resource; number of expected deaths from the disease within the defined group of persons having potential need for the screening test; and data associated with at least one of physical suffering of persons within the group of persons, and psychological suffering of persons within the group of persons, during at least one of at least one additional test of the disease of persons within the group of persons, and treatment of the disease of persons within the group of persons.

According to an embodiment of the system of the present invention, the disease may be caused by a virus.

According to an embodiment of the system of the present invention, the disease may be COVID-19.

According to an embodiment of the system of the present invention, one of the screening tests may be a polymerase chain reaction, PCR, test.

According to an embodiment of the system of the present invention, one of the screening tests may be a lateral flow test.

According to an embodiment of the system of the present invention, the statistical information may further comprise average of an estimated need of at least one additional test to the at least two screening tests, before a final conclusive diagnosis of the disease has been obtained, wherein the input is configured to receive a second level of available at least one additional test to the at least two screening tests, and wherein the system is further configured to select a screening test of the at least two screening tests to be used for the defined group of persons based on the statistical information under a boundary condition that the average of the estimated need of at least one additional test to the at least two screening tests and the average of the estimated need of the at least one treatment resource is not allowed to exceed the first level of availability of the at least one treatment resource, and the second level of available additional tests to the at least two screening test, respectively.

According to an embodiment of the system of the present invention, the average of an estimated need of at least one additional test to the at least two screening tests may include time expenditure of the at least one additional test.

According to an embodiment of the system of the present invention, the disease may be cancer.

According to an embodiment of the system of the present invention, the disease may be prostate cancer.

According to an embodiment of the system of the present invention, one of the screening tests may be a blood test comprising an evaluation of a function of a combination of protein markers, genetic markers and clinical data.

According to an embodiment of the system of the present invention, one of the screening tests may be a blood test which measures the amount of prostate specific antigen, PSA.

Further objectives of, features of, and advantages with, the present invention will become apparent when studying the following detailed disclosure, the drawings and the appended claims. Those skilled in the art will realize that different features of the present invention can be combined to create embodiments other than those described in the following.

### BRIEF DESCRIPTION OF THE DRAWINGS

The invention, together with further objects and advantages thereof, may best be understood by making reference to the following description taken together with the accompanying drawings, in which:

FIG. 1 illustrates schematically a method for management of medical care resources according to an embodiment of the present invention;

FIG. 2 illustrates schematically a classification in a method for management of medical care resources according to an embodiment of the present invention;

FIG. 3 illustrates schematically a step of selecting a screening test of the method for management of medical care resources according to an embodiment of the present invention;

FIG. 4 illustrates schematically screening tests of the method for management of medical care resources according to an embodiment of the present invention.

### DETAILED DESCRIPTION

Throughout the drawings, the same reference numbers are used for similar or corresponding elements.

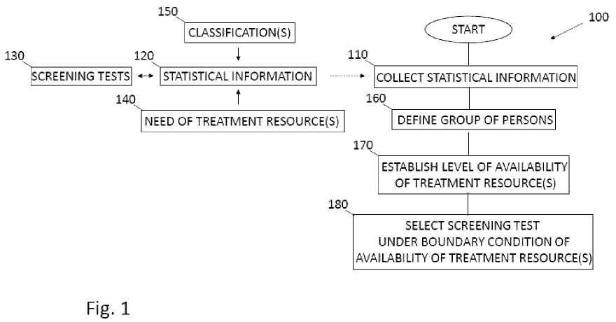

FIG. 1 illustrates schematically a method 100 for management of medical care resources according to an embodiment of the present invention. Examples of medical care resources may comprise medical staff and personnel (e.g. nurses, doctors, etc.), equipment for medical care (e.g. machines, systems, etc.), rooms and/or spaces in hospitals, medical care centers, etc. The method 100 comprises the step 110 of collecting statistical information 120. The statistical information 120 is associated with at least two screening tests 130 for a disease, i.e. tests for identifying one or more conditions of an individual, which condition(s), possibly, could develop into a disease. The screening tests 130 may be substantially any kind of test for screening purposes for substantially any kind of disease. For example, for a disease caused by a virus, such as COVID-19, the screening tests 130 may comprise a polymerase chain reaction (PCR) test, a lateral flow test, etc. According to another example, the disease may be cancer, such as prostate cancer, and the screening tests 130 may comprise blood tests evaluating a function of a combination of protein markers, genetic markers and clinical data, such as the so called Stockholm3® (STHLM3) test, measuring the amount of prostate specific antigen (PSA), etc.

The statistical information 120 comprises an average of an estimated need of at least one treatment resource 140 for treatment of the disease, e.g. including time expenditure of the at least one treatment resource, after a final conclusive diagnosis of the disease has been obtained. The statistical information 120 is associated with at least one classification 150 of an individual of a group of individuals, wherein the classification 150 may comprise a category, characteristic, property and/or attribute of an individual. For example, the classification 150 may comprise or constitute a state of health of the individual (e.g. body mass index, BMI, disease(s), etc. of the individual, the individual being a smoker/non-smoker, etc.) and/or a factor/attribute of the individual such as gender, age, (time of) education, birth country, income, civil status, etc.)

The method 100 further comprises the step of defining 160 a group of persons having an estimated need for one of the at least two screening tests 130 according to the classification(s) 150. In other words, the step of the method comprises defining 160 or selecting a group of persons having an estimated or predicted need for one screening test 130 of the two or more screening tests 130. The method further comprises the step of establishing 170 a first level of availability of the at least one treatment resource. Hence, the step of the method concerns establishing, defining or determining a first level of the availability or disposal of the treatment resource(s). The method further comprises the step of selecting 180 a screening test 130 of the at least two screening tests 130 to be used for the defined group of persons based on the statistical information 120 under a boundary condition that the average of the estimated need of the at least one treatment resource is not allowed to exceed the first level of availability of the at least one treatment resource. In other words, the method comprises the step of selecting 180 a screening test 130 for the defined group of persons based on, or as a function of, the statistical information 120, under the boundary condition that the average of the estimated or predicted need is maintained below, or amounts to, the first level of availability of the treatment resource(s).

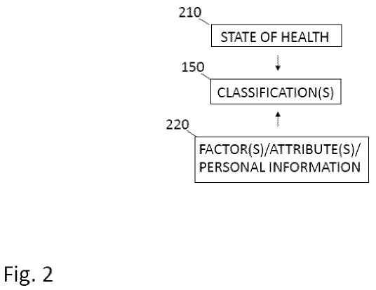

FIG. 2 illustrates schematically a classification 150, as exemplified in FIG. 1, in a method for management of medical care resources according to an embodiment of the present invention. The at least one classification 150 may comprise a state of health 210 of the individual. The state of health 210 may be based on one or more indicators related to the individual's body such as body mass index, BMI, blood pressure, etc. The at least one classification 150 may further comprise at least one factor, attribute, personal information, etc., such as the gender of the individual, the age of the individual, the years of education of the individual, the birth country of the individual, the ethnicity of the individual, the dietary habits of the individual, the income of the individual and the civil status of the individual.

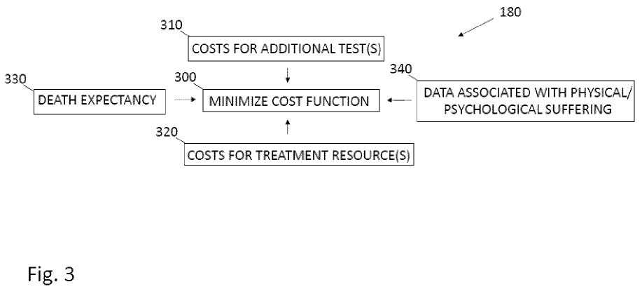

FIG. 3 illustrates schematically a step of selecting 180, as exemplified in FIG. 1, a screening test of the method for management of medical care resources according to an exemplifying embodiment of the present invention. The step of selecting 180 a screening test of at least two screening tests may be performed by minimizing a cost function 300. It will be appreciated that substantially any suitable cost function, in terms of mathematics, may be used, and that details of the cost function 300 therefore is omitted. The cost function 300 may comprise terms being dependent on one or more of the following:
economical costs 310 for at least one additional test of the defined group of persons;
economical costs 320 for the at least one treatment resource;
the number of expected deaths 330 from the disease within the defined group of persons having potential need for the screening test; and
data 340 associated with physical suffering of persons within the defined group of persons and/or psychological suffering of persons within the defined group of persons during at least one additional test of the disease of persons within the defined group of persons and/or treatment of the disease of persons within the defined group of persons. Hence, the step of selecting 180 the screening test may be performed by minimizing the cost function 300, wherein the cost function is dependent on economical costs 310 for one or more additional tests of the defined group of persons and/or for the treatment resource(s) 320, the number of expected deaths 330 from the disease within the defined group of persons having potential need for the screening test, and/or data 340 associated with physical and/or psychological suffering during one or more additional tests and/or treatment of the disease.

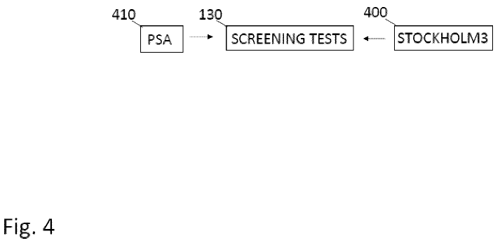

FIG. 4 illustrates schematically screening tests 130, as exemplified in FIG. 1, of the method for management of medical care resources according to an embodiment of the present invention. It will be appreciated that the present invention is particularly advantageous for a disease such as prostate cancer. One of the screening tests 130 for prostate cancer may be a blood test 400 comprising an evaluation of a function of a combination of protein markers, genetic markers and clinical data, also denoted Stockholm3® (or STHLM3). Another screening test 130 for prostate cancer may be a blood test which measures the amount of prostate specific antigen, PSA 410. The Stockholm3® blood test 400 is particularly advantageous in that is may detect (aggressive) prostate cancer at an early stage, wherein early detection is the key to successful treatment of prostate cancer. The PSA blood test 410 is commonly used, but one problem with the PSA test is that it may miss between 30 and 50 percent of all aggressive cases of prostate cancer. In contrast, research has shown that Stockholm3® 400 may find 100 percent more aggressive cases of cancer. Hence, the Stockholm3® blood test 400 may improve the ability to detect and treat prostate cancer at an early stage.

The aspects and embodiments of the present invention presented above may further be illustrated by the following examples.

The method and system according to the present invention may seem non-technical at first glance. While remembering that generic technical solutions in the end aim at providing tools which serve, assist or replace human activities of different kinds, including mental ones, the present invention provides a method and system for the objective selection of a screening method. This is an intricate choice wherein many layers of technical and non-technical elements need to combined. Here, a user is approaching an intellectual task he/she has to master, and the present invention contributes to the technical problem of minimizing spending of medical care resources even if an evaluation by the user on a mental level is involved. In this respect it is the organization of and provision of underlying data in view of a technical problem that matters, and not the mental act seen in isolation.

### Example 1

Recently, the pandemic of COVID-19 has forced health care providers to manage medical care resources in a detailed manner. Testing individuals for COVID-19 to understand prevalence and incidence, to trace disease spread patterns, and to quarantine individuals with confirmed COVID-19 infection is one viable input for managing medical care resources.

There are many types of screening tests for COVID-19, but for the sake of managing care resources, two categories are considered to be better suited than others. Firstly, a PCR screening test is capable of determining presence of genetic material originating from the SAVS-CoV-2 virus (that causes COVID-19) in a sample specimen, typically a nasal swab. Under the assumption that the subject has conducted the nasal swab according to instructions, the PCR test is considered very reliable both in terms of sensitivity and specificity (above 99% in both cases). This example relies on the characteristics of the FDA-CDC SARS-CoV-2 PCR test as supplied by A23 Lab AB in Sweden to Swedish health care in year 2020. The actual measurement of a PCR test requires a full medical laboratory and it takes about 24 hours to produce results (although prioritized samples may get a result in about 4 hours). The cost of a PCR test was, in the early phase of the pandemic, approximately 100 EUR each. PCR testing is considered conclusive with respect to COVID-19 but it does not indicate who is in need of medical resources. Secondly, a lateral flow self-test indicating SARS-CoV-2 antigen material in saliva is capable of determining that the virus is present in the sample specimen. Antigen is a generic term for molecular structures on a pathogen that the immune system recognizes as foreign and potentially harmful. A lateral flow test as a screening test can be made in a doctor's or nurse's office (i.e. no need for a laboratory) and it takes 10-30 minutes to generate a result. This example relies on the characteristics of the Rapid Response® COVID-19 Antigen Rapid Test Cassette (BSV-COV-2C25B) as provided by BioServe UK. This test was used at A23 Lab AB for staff screening after the in-house PCR test for SARS-CoV-2 was discontinued. This particular lateral flow test has approximately 95% sensitivity and near 100% specificity, and it costs about ⅓ of the PCR test. Lateral flow testing is not always considered conclusive in terms of COVID-19, hence a follow-up confirmation with a PCR test may be required. Lateral flow tests do not provide information on the need of medical resources.

Generic statistics related to COVID-19 in a population (like mortality, age distribution and the similar) was obtained from public records at the Swedish National Board of Health and Welfare. The need for an individual, confirmed positive for COVID-19, to have access to a medical care resource may relate to many factors, including age, concomitant diseases, respiratory function (e.g. oxygen saturation), and overall status. The limiting medical resource is intensive care units, and other lesser equipped but specialized hospital wards for COVID-19 patients.

The task for the management of the medical care resources is to, given available screening test capacity, optimize the use of intensive care units and specialized hospital wards. In the early phases of the COVID-19 pandemic, it became clear that elderly people were particularly vulnerable to COVID-19. The intensive care units were filled with patients because it was unknown how to best treat COVID-19 patients. Also, whereas PCR tests were established, the lateral flow tests were difficult to source because production was scaling up.

In the early phase of the pandemic, it would have made sense to define a first group of persons having an estimated need for a screening test and, in particular, for which (frequent) testing would be advisable. For example, elderly (e.g. aged 70 and above) and all support staff that are in frequent contact with elderly (hospital staff, care home staff and the similar) could constitute such a group of persons. Hence, there would be a defining of a group of persons having an estimated need for one of the at least two screening tests according to a classification according to the method of the present invention. Since the elderly occupied a significant fraction of the intensive care unit resources and since the mortality of elderly with COVID-19 was in the order of 25% in the early phase of the pandemic, reducing the number of COVID-19 infections among elderly would have a direct effect on resource use of the care institutions such as hospitals. The screening test of choice should be PCR, because the effect of the lesser sensitivity of lateral flow testing means that a staff at an elderly care home with a false negative lateral flow test result may result in a complete disease transmission within the care home, potentially affecting many elderly with the pain and human suffering in parallel to massive care resource spending.

In later phases of the pandemic, it would make sense to define a second group of persons having an estimated need for a screening test, according to the classification according to the method of the present invention. For example, staff at society critical functions (e.g. hospitals, banking sector, fire brigade, police, and the similar) could constitute this second group of persons. This group of persons is predominantly aged between 25 and 65 years, an age group with lower mortality of COVID-19 but also an age group comprising a larger number of individuals. The main objective would hence be to reduce the need for specialized hospital ward resources while maintaining societal function. It would be advisable to subject this second group to frequent testing. A lateral flow test would be acceptable because the mortality is low in this age group and the cost is lower.

### Example 2

Prostate cancer is one of the most common cancer forms in men in the Western world. The prostate cancer diagnostic chain typically comprises a series of tests and examinations, each refining the available information. A first screening step is typically conducted in which a blood test is subjected to analysis for elevated risk for prostate cancer.

A suitable option for a screening step is a blood test comprising an evaluation of a function of a combination of protein markers, genetic markers and clinical data, according to an embodiment of the present invention, such as the so called Stockholm3® (or STHLM3) test (Prostate cancer screening in men aged 50-69 years (STHLM3): a prospective population-based diagnostic study, Henrik Grönberg et al, Lancet Oncol 2015, Published Online Nov. 9, 2015 http://dx.doi.org/10.1016/S1470-2045(15)00361-7). This Stockholm3® screening test is multiparametric and combines the results of many measurements into a risk score. The Stockholm3® risk score has been shown to estimate risk in a much more correct manner than current clinical practice (prostate specific antigen (PSA) testing), reducing the negative biopsies with about 50% while resulting in the same number of found cancers as clinical practice would (Eklund M, et al. The Stockholm-3 (STHLM3) Model can Improve Prostate Cancer Diagnostics in Men Aged 50-69 yr Compared with Current Prostate Cancer Testing. Eur Urol Focus (2016), http://dx.doi.org/10.1016/j.euf.2016.10.009).

One piece of information related to the Stockholm3® test is related to mortality. The study participants in the original study conducted 2013-2015 along with other men in the same age group living in the same region were followed up after 5 years. Among the individuals that were invited and participated in the Stockholm3® study and were screened with a single Stockholm3® test, 15 out of 58502 men died of prostate cancer, corresponding to 2.56*10\hat{ }−4 or 0.0256%. Among the men that were invited but declined to participate in the Stockholm3® study (and accordingly did not get any Stockholm3® test), 59 of 115348 men died of prostate cancer within 5 years, corresponding to 5.11*10\hat{ }−4 or 0.0511%. In the same region, there were also 74561 men that were not invited to the study at the time. In that group, 50 men died of prostate cancer within 5 years, corresponding to 6.71*10\hat{ }−4 or 0.0671%.

Hence, it is concluded that screening for prostate cancer with Stockholm3® brings a benefit in that unnecessary biopsies are heavily reduced (as previously published), according to the subject-matter of an embodiment of the invention of an estimated need of at least one additional test to the at least two screening tests before a final conclusive diagnosis of the disease has been obtained. Furthermore, the results of the Stockholm3® test makes it possible for health care providers to manage medical resources (such as treatment) in a manner that reduces mortality to about half in 5 years.

### Example 3

It should be noted that when a tested man is deemed at elevated risk for prostate cancer, this is typically followed by a series of increasingly expensive investigations. A magnetic resonance imaging step may be conducted to determine if there are abnormal structures in the prostate (putatively a prostate cancer tumor). Thereafter, a biopsy of the prostate may be conducted, where about 12 needles are used to harvest prostate material which is subjected to pathology analysis to determine presence of prostate cancer cells. The biopsy result is usually considered a truthful and confirmative diagnosis, in turn leading to the use of costly medical resources like radiation therapy or surgery. It should be noted that prostate cancer is a very slowly progressing disease, to the level where national and international guidelines advocate against screening and treatment for men with less than 15 years expected lifetime. Prostate cancer is further uncommon in men aged below 50 years.

There are different options available for a (first) screening step for prostate cancer. One option is to use prostate specific antigen (PSA) testing, which is current clinical practice. Whereas PSA is known to be elevated in men with prostate cancer, it is also known to be a gland specific test. This means that an elevated PSA value can be due to a multitude of factors, including infection, inflammation, physical exercise, and cancer. The specificity of the PSA testing is hence low, and the number of false positive results from PSA testing often exceeds 50%. PSA testing per se is however inexpensive, approximately 20 EUR per test. From an overview standpoint, it is known (https://jamanetwork.com/journals/jama/fullarticle/2673968, Martin and co-authors, JAMA. 2018; 319(9):883-895. doi: 10.1001/jama.2018.0154) that a single PSA test does not reduce prostate cancer mortality after a median follow-up time of 10 years.

Another option for the screening test is the Stockholm3® test, discussed in Example 2. The cost of a Stockholm3® test is approximately 10 times higher than a PSA test. The task for the management of medical care resources is to, given available screening test capacity, optimize the use of scarce pathology resources (for biopsy evaluation) and expensive treatment resources (like radiation therapy, surgery) while keeping the population healthy. It would make sense to define a group of persons having an estimated need for a prostate cancer screening test, e.g. men aged 50-70 years. This group of persons has long enough expected lifetime to warrant medical action if a prostate cancer is confirmed, and are of an age where prostate cancer incidence is elevated. For this group of persons, it would make sense to use a screening test that minimizes the spending of medical resources (like pathology and treatment resources) while finding relevant cancers. The cost of the screening test becomes less important because the savings in medical resource spending may become significant. On top of the direct savings of medical resources, should the screening test that allow for management of care resources in a manner that extend the lifetime of men, savings for society both in terms of human suffering and in terms of economy are even bigger. Stockholm3® has been shown to save resources compared to PSA (Eirik Viste, Cathrine Alvaer Vinje, Torgeir Gilje Lid, Svein Skeie, Øystein Evjen-Olsen, Tobias Nordström, Olav Thorsen, Bjørnar Gilje, Emiel A. M. Janssen & Svein R. Kjosavik (2020) Effects of replacing PSA with Stockholm3® for diagnosis of clinically significant prostate cancer in a healthcare system—the Stavanger experience, Scandinavian Journal of Primary Health Care, 38:3, 315-322, DOI: 10.1080/02813432.2020.1802139), where the saving was estimated to 340 Euro per man. Hence there is publicly available indications that, compared to PSA, there is a direct saving of about 340 Euro per man.

Stockholm3® has also been shown to reduce mortality (Example 2). Assigning approximately 250 000 Euro as the value of a year (Läkartidningen. 2018,115:E96F https://lakartidningen.se/opinion/debatt/2018/08/ett-galy-ar-vart-mer-an-tva-miljoner-kronor/) and assuming that each survivor due to the Stockholm3® screening strategy (compared to PSA) gain 5 years of additional life leads to important additional savings, both in terms of direct operational savings and extended life time savings. Hence, Stockholm3® could, in comparison to PSA, induce both operational direct savings and extended life time savings, which in turn would render Stockholm3® a strong candidate for a screening test in this scenario.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A method for management of medical care resources, comprising the steps of:
collecting statistical information associated with at least two screening tests for a disease;
the statistical information comprising:
average of an estimated need of at least one treatment resource for treatment of the disease, after a final conclusive diagnosis of the disease has been obtained;
the statistical information being associated with at least one classification of an individual of a group of individuals;
defining a group of persons having an estimated need for one of the at least two screening tests according to the classification;
establishing a first level of availability of the at least one treatment resource;
selecting a screening test of the at least two screening tests to be used for the defined group of persons based on the statistical information under a boundary condition that the average of the estimated need of the at least one treatment resource is not allowed to exceed the first level of availability of the at least one treatment resource.

2. The method of claim 1, wherein the average of an estimated need of at least one treatment resource for treatment of the disease includes time expenditure of the at least one treatment resource.

3. The method of claim 1, wherein the at least one classification comprises at least one of
a state of health of the individual, or
at least one factor selected from the group consisting of the gender of the individual, the age of the individual, the years of education of the individual, the birth country of the individual, the ethnicity of the individual, the dietary habits of the individual, the income of the individual, and the civil status of the individual.

4. The method of claim 1, wherein the at least one treatment resource comprises at least one of
medical staff for treatment of the defined group of persons,
medical equipment for treatment of the defined group of persons, or
pharmaceuticals for treatment of the defined group of persons.

5. The method of claim 1, wherein the step of selecting a screening test of the at least two screening tests is performed by minimizing a cost function, the cost function comprising terms being dependent on at least one of:
economical costs for at least one additional test of the defined group of persons;
economical costs for the at least one treatment resource;
number of expected deaths from the disease within the defined group of persons having potential need for the screening test; or
data associated with at least one of
physical suffering of persons within the defined group of persons, or
psychological suffering of persons within the defined group of persons,
during at least one of
at least one additional test of the disease of persons within the defined group of persons, or
treatment of the disease of persons within the defined group of persons.

6. The method of claim 1, wherein the disease is caused by a virus.

7. The method of claim 1, wherein the disease is COVID-19.

8. The method of claim 6, wherein one of the screening tests is a polymerase chain reaction (PCR) test.

9. The method of claim 6, wherein one of the screening tests is a lateral flow test.

10. The method of claim 1, wherein the statistical information further comprises
average of an estimated need of at least one additional test to the at least two screening tests before a final conclusive diagnosis of the disease has been obtained,
and wherein the method further comprises the steps of
establishing a second level of available at least one additional test to the at least two screening tests, and
selecting a screening test of the at least two screening tests to be used for the defined group of persons based on the statistical information under a boundary condition that the average of the estimated need of at least one additional test to the at least two screening tests and the average of the estimated need of the at least one treatment resource is not allowed to exceed the first level of availability of the at least one treatment resource, and the second level of available additional tests to the at least two screening test, respectively.

11. The method of claim 10, wherein the average of an estimated need of at least one additional test to the at least two screening tests includes time expenditure of the at least one additional test.

12. The method of claim 10, wherein the disease is cancer.

13. The method of claim 10, wherein the disease is prostate cancer.

14. The method of claim 12, wherein one of the screening tests is a blood test comprising an evaluation of a function of a combination of protein markers, genetic markers and clinical data.

15. The method of claim 12, wherein one of the screening tests is a blood test which measures the amount of prostate specific antigen, PSA.

16. A system for management of medical care resources, comprising
an input configured to receive
statistical information associated with at least two screening tests for a disease, the statistical information comprising average of an estimated need of at least one treatment resource for treatment of the disease, after a final conclusive diagnosis of the disease has been obtained, wherein the statistical information is associated with at least one classification of an individual of a group of individuals,
information associated with a group of persons having an estimated need for one of the at least two screening tests according to the classification, and
a first level of availability of the at least one treatment resource,
wherein the system is configured to select a screening test of the at least two screening tests based on the statistical information under a boundary condition that the average of the estimated need of the at least one treatment resource is not allowed to exceed the first level of availability of the at least one treatment resource.

17. (canceled)

18. The system of claim 16, wherein the at least one classification comprises at least one of
a state of health of the individual, or
at least one factor selected from the group consisting of the gender of the individual, the age of the individual, the years of education of the individual, the birth country of the individual, the ethnicity of the individual, the dietary habits of the individual, the income of the individual, and the civil status of the individual.

19. (canceled)

20. The system of claim 16, wherein the system is further configured to select a screening test of the at least two screening tests by minimizing a cost function, the cost function comprising terms being dependent on at least one of:
economical costs for at least one additional test of the defined group of persons;
economical costs for the at least one treatment resource;
number of expected deaths from the disease within the defined group of persons having potential need for the screening test; or
data associated with at least one of
physical suffering of persons within the group of persons, or
psychological suffering of persons within the group of persons, during at least one of at least one additional test of the disease of persons within the group of persons, or
treatment of the disease of persons within the group of persons.

21. (canceled)

22. The system of claim 16, wherein the disease is COVID-19.

23-24. (canceled)

25. The system of claim 16, wherein the statistical information further comprises
average of an estimated need of at least one additional test to the at least two screening tests before a final conclusive diagnosis of the disease has been obtained,
wherein the input is configured to receive
a second level of available at least one additional test to the at least two screening tests, and
wherein the system is further configured to select a screening test of the at least two screening tests to be used for the defined group of persons based on the statistical information under a boundary condition that the average of the estimated need of at least one additional test to the at least two screening tests and the average of the estimated need of the at least one treatment resource is not allowed to exceed the first level of availability of the at least one treatment resource, and the second level of available additional tests to the at least two screening test, respectively.

26-30. (canceled)
